(19) United States
(12) Patent Application Publication
(10) Pub. No.: US 2008/0319472 A1
(43) Pub. Date: Dec. 25, 2008

(54) Cervical dilator catheter

(76) Inventor: Marion Stevens Shelley, Hope Mills, NC (US)

Correspondence Address: Marion Stevens Shelley, 6940 County Place Drive, Hope Mills, NC, 28348, US

(21) Appl. No.: 11/820,196

(22) Filed: Jun. 19, 2007

(51) Int. Cl. A61M 29/02

(52) U.S. Cl. 606/193

(57) ABSTRACT

This invention is an improved balloon catheter for cervical dilation. This catheter has a balloon that is passed through the cervix of a pregnant woman, and the balloon is then inflated with 100-120 ml of sterile water or normal saline. The improved disc shape of the inflated balloon is 8 cm in diameter and 3-4 cm in height. The balloon will sweep the amniotic membrane away from the cervix, causing the release of prostaglandins which helps ripen the cervix and initiates uterine contractions. The improved shape of the balloon causes the cervix to be 6-8 cm dilated once it is expelled. This catheter also contains a flexible guidewire that allows for easier insertion for the provider, and makes insertion less painful for the patient because it can be placed without a speculum and ring forcep being used. This catheter could actually be placed on an outpatient basis or in the doctor's office, with the patient instructed to go to the hospital or birthing unit when the catheter falls out. This catheter is smaller in diameter than the 16 French Foley catheter, so therefore it can be placed when the patient is <1 cm dilated. This catheter when placed properly and secured to the patient's thigh with tension applied can shorten labor by 4-10 hours.

### BACKGROUND OF THE INVENTION

Induction of labor has always been dependant on the favorability of the cervix to dilate. In order to induce labor many different medications and devices are currently utilized to bring about cervical dilation. The most widely used method is intravenous administration of oxytocin. This method is often a prolonged process, especially if the cervix is unfavorable for dilation. It also requires the patient to be on continuous fetal monitoring which limits the patient's mobility. It involves patient discomfort and can cause uterine hyperstimulation and fetal distress. Another method is the administration of cytotec to the cervix, however the results can be unpredictable. It can lead to uterine hyperstimulation and uterine rupture, which places the baby and the mother at risk for death. Another drawback is that it is difficult to remove from the cervix if hyperstimulation does occur.

Other methods used to ripen the cervix include prostaglandin gels applied to the cervix which also can take many hours to work as well as cause various unwanted side effects such as nausea, vomiting, diarrhea, dysrhythmias and uterine hypertonus. Laminara has also been used to ripen the cervix, but it is uncomfortable and unreliable, and it can also take many hours for it to ripen the cervix so that the induction may be started. Stripping the membranes is another method that is used rather frequently to initiate labor. This is done by the provider inserting a gloved finger into the cervix and sweeping the amniotic membrane away from the cervix, thus causing the release of prostaglandins. This can initiate uterine contractions and help start labor, however the patient must be 1-2 cms dilated in order for this to done.

Mechanical dilators such as the Foley catheter with a 30 ml balloon or an ATAD catheter U.S. Pat. No. 4,976,692 have been used with good success. Especially, the Foley catheter with the 30 ml balloon. This catheter is placed into the cervix and once the balloon is just past the cervix then it is inflated with 50-60 ml of sterile water or normal saline. The catheter is then pulled tight and secured to the patient's thigh, and when it falls out the patient is 4-5 cms dilated. However these catheters can be uncomfortable to have placed, due to most providers using a speculum to visualize the cervix and then they thread the catheter in with a pair or ring forceps. Also the patient usually must be at least 1-2 cms dilated in order for them to be placed. Another disadvantage is that no matter how much more water is placed in the balloon, it will only have a diameter of 4-5 cms. These catheters have also been placed and a slow infusion of saline through the catheter has been used to continuously sweep the membranes away from the cervix. Although this can be risky if the catheter tip becomes clogged because the saline can build up causing an abruption.

Thus, there is the need for a catheter that can be placed much easier, and more comfortably for the patient, but also one that is simple in its method of action, safe and one that can shorten the length of labor. This could significantly lower the number of cesarean deliveries done because of the cervix's failure to dilate.

### SUMMARY OF THE INVENTION

The present invention provides an improved single balloon catheter that is intended to dilate the cervix, initiate uterine contractions, and therefore shorten labor. It is approximately 42 cm in length and 2.5 mm to 4 mm in diameter. It has a balloon on the distal end, just below the drainage outlet, that when inflated it has a disc shape with a diameter of 6 cm to 8 cm and a height of 3 cm to 4 cm. The total volume should be about 100 ml to 120 ml.

The balloon, inflation tube and catheter shaft are preferably constructed of biocompatible materials. These materials include, but are not limited to, silicone, rubber including nitrile rubber, styrene butadiene rubber, viton, or butyl rubber, silicon elastomer, Dacron-reinforced silicon material, latex, polyethylene, Mylar, or Teflon. The material for the balloon should be elastomeric in nature to allow for expansion and contraction of the balloon as it is inflated or deflated.

The catheter shaft is an elongated tube that will accommodate a flexible, removable plastic stylet. The stylet has the properties of being flexible yet resilient enough to stiffen the catheter to make insertion through the cervix easier. The catheter shaft has a drainage outlet, on the side at the distal end, which allows for drainage of amniotic fluid in case of rupture of the amniotic sac. The catheter shaft also contains a smaller inflation tube inside, that has an injection port whereas a syringe can be connected to inflate the balloon with sterile water or normal saline.

Prior to inflation of the balloon the distal end of the catheter is placed through the undilated or partially dilated (0-1 cm) cervix with the stylet in place to guide the catheter through the cervix. Once the distal end with the balloon is through the cervix and in the uterine region, a syringe is utilized to inject 100 to 120 ml of sterile water or normal saline into the balloon. As the balloon is inflated it lifts the amniotic membrane off of the cervix, therefore sweeping the membrane which causes the release of prostaglandins which initiate cervical ripening and uterine contractions. The stylet is then removed, and the catheter is pulled taut and secured with adhesive tape to the patients' thigh. This causes a downward pressure on the cervix that imitates the fetal head. The patient can then walk which would provide continual stimulation of the cervix causing uterine contractions or oxytocin can be administered intravenously with the catheter being tugged gently at regular intervals when patient ambulation is not possible. When the catheter is expelled, the patient will be 6 cm to 8 cm dilated. This invention could shorten the length of labor significantly, therefore decreasing the number of ceasarean deliveries due to failure to dilate.

### BRIEF DESCRIPTION OF THE DRAWINGS

The invention is herein described, by way of example only, with reference to the accompanying drawings, wherein:

FIG. 1 illustrates one form of the catheter constructed in accordance with the present invention, when the balloon is deflated; and

FIG. 2 illustrates the catheter constructed in accordance with the present invention, with the balloon inflated with 100-120 ml of sterile water or normal saline; and

FIG. 3 is a diagram illustrating the manner of using the catheter in FIG. 1 for inducing labor.

### DESCRIPTION OF ILLUSTRATIVE EMBODIMENTS

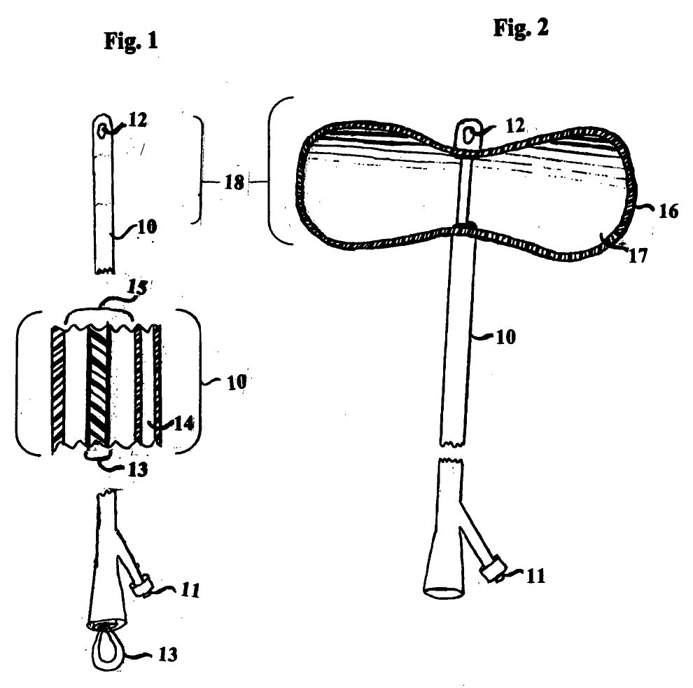

The catheter as illustrated in FIG. 1 comprises a slender, flexible tube 10 that has an outlet port 12 on the side of the distal tip, this allows for drainage of amniotic fluid through the main lumen 15 of the catheter. There is a fixed inflatable balloon 16 on the distal end as shown in FIG. 1 and FIG. 2. This balloon 16 is inflated with sterile water 17 or normal saline 17 as shown in FIG. 2. The liquid is introduced into the balloon 16 through an injection port 11 and through the inflation tube 14. The balloon 16 is disc-shaped, measuring approximately 3-4 cm in height with a diameter of 6-8 cm when inflated as shown in FIG. 2. The catheter 10 also accommodates a flexible, bendable, removable plastic stylet 13 in the main lumen 15 of the catheter, as shown in FIG. 1.

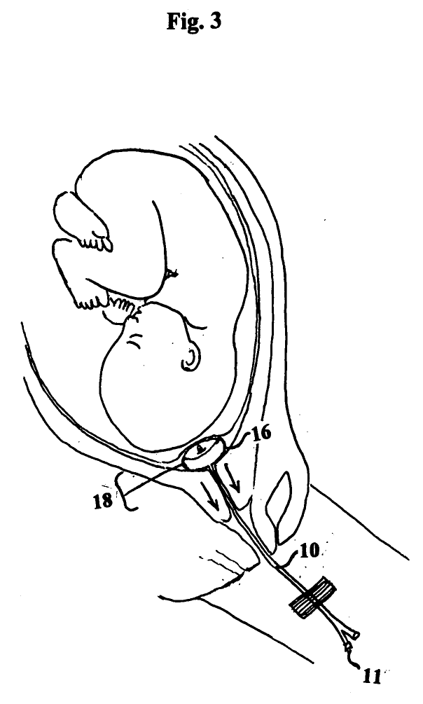

FIG. 3 illustrates placement of the catheter of the catheter 10 in the cervical region of the uterus for use as a cervical dilator. With the balloon 16 deflated, and with the stylet 13 in place the provider inserts the distal end 18 (FIGS. 1 and 2) of the catheter through the undilated or partially dilated cervix. Once the distal end 18 is past the cervix then a syringe with normal saline 17 or sterile water 17 is injected through the injection port 11 and through the inflation tube 14 into the balloon 16. Inflating the balloon 16 causes the amniotic membranes to be swept off of the cervix, causing the release of prostaglandins. The stylet 13 is then removed and discarded. The provider then pulls the catheter 10 taut against the cervix and secures it to the patient's thigh with tape, thereby providing downward pressure on the cervix causing it to ripen and dilate.

I claim:

1. An improved balloon catheter particularly useful for application to the cervix of the uterus in order to ripen and dilate the cervix, comprising: a slender flexible tube that has outlet openings on the side of the distal tip; an inflatable balloon fixed to the tube just below the outlet opening on the distal end; an inflation tube inside the main tube that allows for fluid to be injected into the balloon by syringe through an injection port on the proximal end of the inflation tube; an flexible, removable plastic stylet that is in the main lumen of the catheter to assist with guiding the catheter through the cervix.

2. The catheter according to claim 1 wherein the catheter has an approximate length of 42 cm and a approximate diameter of 2.5-4 mm when the balloon is deflated.

3. The catheter according to claim 1 wherein the inflatable balloon has an approximate diameter of 6-8 cm and a height of approximately 3-4 cm when it is inflated with a total volume of 100-120 ml of sterile water or normal saline.

4. The catheter according to claim 1 wherein it is placed through the closed to partially dilated cervix and into the uterine region, with the balloon then being inflated with 100-120 ml of sterile water or normal saline, thereby sweeping the amniotic membrane away from the cervix causing the release of prostaglandins, which helps initiate labor and ripens the cervix.

5. The catheter according to claim 1 wherein the inflated balloon is pulled taut against the cervix and secured to the patient's thigh, causing a downward pressure on the cervix which promotes cervical dilation.

6. The catheter in claim 1 wherein upon it being expelled the patient will be dilated 6-8 cms.

7. The catheter in claim 1 wherein the flexible, removable, plastic stylet is resilient enough to make the catheter firm enough to be guided easily through the cervix without the need for a speculum or ring forcep, making it easier to be placed for the provider and less painful for the patient.

8. The catheter in claim 1 wherein it's placement and mechanism of action is simple enough that it can be placed on an outpatient or office type visit with the patient being instructed to go to the hospital upon expulsion of the catheter.

9. The catheter in claim 1 wherein will shorten labor by 4-8 hours.

10. The catheter in claim 1 can be removed easily if complications arise.

11. The catheter in claim 1 may decrease the number of cesarean deliveries done due to failure to dilate because the cervix was unfavorable for induction of labor.

12. The catheter in claim 1 wherein allows for the patient to walk or move around while the catheter ripens and dilates the cervix.
